(19) United States
(12) Patent Application Publication
(10) Pub. No.: US 2007/0055259 A1
(43) Pub. Date: Mar. 8, 2007

(54) Apparatus and methods for removal of intervertebral disc tissues

(76) Inventors: Britt K. Norton, Eden Prairie, MN (US); Christine M. Horton, Prior Lake, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/207,094

(22) Filed: Aug. 17, 2005

(51) Int. Cl. A61B 17/00

(52) U.S. Cl. 606/79

(57) ABSTRACT

Apparatus and methods for removing tissue from an intervertebral disc are disclosed. The apparatus can include an elongated guide tube, a rotary cutting member and a drive shaft. Other apparatus can include an elongated guide tube, an inner guide tube, a cutting head, a rotary cutting member and a drive shaft. The apparatus are generally configured to extend and withdraw a rotary cutting member or a rotary cutting member in combination with a cutting head from and into the distal end of the elongated guide tube to cut and/or abrade tissues within an intervertebral disc.

### BACKGROUND OF THE INVENTION

1. Field of the Invention

The present invention relates to removal of intervertebral discs and, more particularly, to apparatus and methods for removal of the nucleus pulposus of an intervertebral disc.

2. Description of the Related Art

The spine is made up of twenty-four bony vertebrae, each separated by a disc that both connects the vertebrae and provides cushioning between them. The lumbar portion of the spine has five vertebrae, the last of which connects to the sacrum. The disc is comprised of the annulus, which is a tough, layered ligamentous ring of tissue that connects the vertebrae together, and the nucleus, a gelatinous material that absorbs water and is fed through the endplates of the vertebrae. In a healthy disc, the nucleus is pressurized within the annulus much like the air is pressurized within an automobile tire.

Degenerative disc disease (DDD) is a condition that affects both structures of the disc, and is usually thought of as a cascade of events. In general, DDD is characterized by a weakening of the annulus and permanent changes in the nucleus, and may be caused by extreme stresses on the spine, poor tone of the surrounding muscles, poor nutrition, smoking, or other factors. In DDD, the nutrient flow to the nucleus is disrupted and the nucleus loses water content. As the nucleus dehydrates it loses pressure, resulting in a loss of disc height and a loss in the stability of that segment of the spine. In the lumbar spine, as the degenerative cascade continues, the annulus may bulge and press on a nerve root, causing sciatica (leg pain) among other problems. The loss of disc height can also result in leg pain by reducing the size of the opening for the nerve root through the bony structures of the spine. As the disc loses height, the layers of the annulus can begin to separate, irritating the nerves in the annulus and resulting in back pain.

Surgical treatment for early DDD, where the pain is primarily leg pain, is usually a discectomy where some the nucleus material is removed to reduce the bulging of the disc and the pressure on the nerve root. For more severe cases of DDD, where the disc has completely collapsed and/or where a discectomy did not have long-term success, the traditional surgical treatment has been fusion of the vertebrae through the use of plates, rods, pedical screws, and interbody fusion devices. For years, surgeons and industry have been looking for ways to interrupt the degenerative cascade for patients with early stage disease, and for methods that retain motion at the affected disc in patients with more advanced disease. Just as the surgical treatment for degenerated knees and hips changed from fusion to motion preservation (arthrodesis to arthroplasty), innovative technologies are now creating a market for treatment of DDD without resorting to fusion. The field of spinal arthroplasty represents a significant emerging market in spinal surgery.

Surgical treatment for early stage disease that involves primarily leg pain as a result of a herniated disc is currently limited to a simple discectomy, where a small portion of the disc nucleus is removed to reduce pressure on the nerve root, the cause of the leg pain. While this procedure is usually immediately successful, it offers no means to prevent further degeneration, and a subsequent herniation requiring surgery will occur in about 15% of these patients.

A range of prosthetic techniques has been developed and continue to be developed for the treatment of DDD. These techniques typically use one of three types of prosthetic devices: total disc replacement (TDR) devices, which sacrifice much of the connective tissue of the disc and are intended for discs with severe degeneration; partial disc replacement (PDR) devices, which replace only the nucleus of the disc; and flexible springs and connectors attached to the posterior bony elements of the spine. The PDR will be marketed as the surgical treatment of choice for patients with slightly more advanced (mild-to-moderate) disc degeneration. This technology relies on the connective structures of the affected level, such as the annulus, facets, and longitudinal ligaments, to be relatively healthy. A fourth type of device, used for repairing the annulus after a herniation or implantation of a PDR, is also currently in development.

Current designs for nucleus replacement devices are typically not attached to the nucleus or vertebra, and are free to move within the nucleus cavity. Much like the healthy nucleus, these devices are subjected to the high forces and the twisting and bending motions that must be endured by the spinal structures, and some device movement is expected. Current PDR devices have a known complication of excessive device movement, however, and can move back out the annulus at the site of implantation. This device extrusion can occur in over 25% of cases for some designs. While the effect of the complication is not life threatening, the response is another surgery to reposition or replace the PDR, or to remove it altogether and likely replace it with a total disc replacement or a fusion procedure. There is mounting evidence that the nucleus material left in the disc cavity, even after an exhaustive removal procedure, can push against even a well-positioned PDR and be the cause of many of the device extrusions. When a posterior approach is used for removal, the remaining nucleus material left behind can push against a PDR. While more of this material could be removed if the disc is accessed via a lateral or an anterior approach, current information indicates that most spine surgeons prefer to use the posterior approach.

The annulus repair technologies that rely on mechanical means to close the annulus involve the need to contact and/or secure to the inside of the annulus tissue immediately adjacent to the site used to access the nucleus cavity. These designs will achieve the best deployment and surgical attachment to the annulus if the bulk of the relatively soft nucleus material near the access site has been adequately removed. Remaining nucleus material can have a negative impact on the performance of these devices if it is not removed. This material will be difficult to remove whether the access to the cavity is performed via a posterior, lateral, or an anterior surgical approach.

For annulus repair and PDR, among other procedures, implantation site preparation typically involves removal of the nucleus. A wide range of devices have been developed for this removal procedure. However, surgeons have historically utilized an array of pituitary rongeurs for the various procedures requiring removal of the nucleus pulposus or portions of the nucleus pulposus.

The rongeur is provided in a variety of configurations including “up-biting”;

straight; and “down-biting”, and can be found in a variety of lengths, widths, and with razor or serrated jaws. However, even using the preferred posterior access to the disc with a rongeur, its useful range of motion within the intervertebral disc is limited. The bony structure of the posterior spinal elements, even though partially removed to provide access for PDR implantation, typically limits the angles through which the rongeur can be maneuvered. This limitation of movement serves to limit the amount of nucleus material that can be removed. More importantly, the limitation on movement may not allow adequate removal of material next to the annular access to provide good contact for an annular repair device and does not allow adequate removal of material contralateral to the annular access, preventing optimal position for a PDR. Further, the use of a rongeur requires constant insertion and removal to clean the nucleus material from the tip of the device, resulting in dozens of insertion/removal steps to remove an adequate amount of material from the nucleus. This can increase the trauma to the surrounding annulus tissue and increase the risk of damaging the endplates.

An additional significant limitation of the rongeur instrument is the ability to easily remove the important annular tissue, especially when using rongeurs with a sharp cutting tip. Surgeons typically do not try to remove the entire nucleus in simple discectomy procedures, or intentionally remove annulus in preparation for fusion procedures. In this respect, a surgeon's “feel” for the tissue, or ability to distinguish softer nucleus tissue from tougher annulus tissue, may not be well developed and PDR site preparation may result in significant trauma to the annulus.

A range of more sophisticated devices for removing nucleus has been developed, however, the adoption of these devices has been very limited. Some of the more intricate devices utilize mechanized cutting mechanisms for removal of material from the nucleus pulposus. Frequently, these devices require suction and/or irrigation to remove material during the procedure.

One device uses a guillotine-style assembly that cuts nucleus material, aspirates the material into the instrument tip, and then evacuates the cut material is through the instrument. Movement of the guillotine assembly is automated and controlled by a mechanism in the handpiece of the instrument. The continuous removal of tissue without the need to repeatedly insert and remove the instrument minimizes trauma to the surrounding tissue. The guillotine type assembly is typically associated with a straight, stiff device, that is intended for a minimally invasive, percutaneous approach. Because of their stiffness, although the devices may be somewhat effective for a lateral or anterior surgical approach for PDR implantation, they are generally not usable for nucleus removal utilizing a posterior approach.

Other devices have utilized an Archimedes type screw to pull nucleus material into the catheter and shear it when it reaches the tip of the catheter. Continued collection of nucleus material by the rotating Archimedes type screw pushes the sheared material through the catheter and into a collection chamber. While less complicated to use than the previously discussed guillotine type assembly, the devices utilizing the Archimedes type screw typically have the similar maneuverability disadvantages. Further, these devices can relatively easily be directed into and through the annulus of the intervertebral disc being treated.

Still other systems have used a high-pressure stream of water to remove nucleus material. In one device, the high-pressure stream of water produces a vacuum which pulls nucleus material into the stream. The high-pressure stream of water then cuts the nucleus material and pulls the material through a catheter to a collection bottle. Among other disadvantages, such systems are expensive. Further, although the tip of the instrument can be bent slightly, its lateral reach when used via the posterior approach is still very limited. Further, since the water stream is very narrow, successful nucleus removal can be technique dependent and time consuming.

Still other devices utilize radio frequency (RF) energy or plasma directed through electrodes for tissue resection and vessel cauterization in preparation for implanting a PDR. These devices typically include an RF generator that can be used with a variety of different types and shapes of electrodes. These devices are typically stiff and have little lateral reach when used making them relatively ineffective for use through the posterior approach. Further, the RF ablation technology can resect annulus or endplate cartilage as easily as nucleus material.

Still other devices utilize lasers to remove material from the nucleus pulposus. These lasers are typically transmitted through a laser fiber positioned within a multi-lumen catheter. These multi-lumen catheters have also included additional components such as imaging fibers, illumination fibers, and irrigation ports. Further, the tip of these catheters can be steerable. Although steerable, the bend radius of the catheters typically prevents them from being useful for removing nucleus near the annulus access. Accordingly, these devices have limited utility for removal of material in preparation for implantation of annulus repair devices. Further, the effective radius of laser beam from these devices is typically only 0.5 mm, making removal of large amounts of nucleus very difficult and time consuming. Detrimentally, lasers can resect annulus or endplate cartilage as easily as nucleus material. Since the tip of the catheter is typically not protected, the laser beam has the ability to easily penetrate and damage the annulus and endplate tissue.

Other devices for nucleus removal are also available. However, these technologies possess their own limitations for the unique needs of annulus repair and PDR device site preparation. The limitations of these devices, along with those of the pituitary rongeur, are driving the need for a more advanced instrument for nucleus removal.

### SUMMARY OF THE INVENTION

Apparatus and methods in accordance with the present invention may resolve many of the needs and shortcomings discussed above and will provide additional improvements and advantages as will be recognized by those skilled in the art upon review of the present disclosure.

In one exemplary embodiment, the present invention may provide an apparatus for removing tissue from an intervertebral disc including an elongated guide catheter, a rotary cutting member and a drive shaft. The elongated guide tube may define a lumen extending from a proximal opening at a proximal end of the elongated guide tube to a distal opening at a distal end of the elongated guide tube. The lumen may include a bend at the distal end of the elongated guide tube. The bend may direct the lumen and the distal opening laterally from the longitudinal axis of the elongated guide tube. The lumen can extend linearly over a linear section extending between the bend and the distal opening. The rotary cutting member may be slidably received within the distal opening at the distal end of the elongated guide tube. The rotary cutting member may be composed of a plurality of filaments configured to cut or abrade tissue. The filaments may have a cross-sectional shape that is round, square, rectangular, parallelogram or other shape as will be recognized by those skilled in the art. The filaments have first ends and second ends. On their second ends, the filaments may include a filament cap. The rotary cutting member may also or alternatively include a plurality of blades to cut or abrade tissue. Further, the rotary cutting member may include an end cap configured to pass through the tissue of the nucleus pulposus but to be only atraumatic to the tissue of the annulus fibrosus. The drive shaft may be rotatably received within the lumen of the elongated guide tube. The drive shaft may extend between the proximal opening at the proximal end of the elongated guide tube and the distal opening at the distal end of the elongated guide tube. The drive shaft is typically connected to the rotary cutting member to confer a rotational force to the rotary cutting member. The lumen of the linear section of the elongated guide tube may be generally configured to direct the rotary cutting member along an axis defined by the linear section of the elongated guide tube.

In another exemplary embodiment, the present invention may provide an apparatus for removing tissue from an intervertebral disc including an elongated guide tube, an inner guide tube, a cutting head, a rotary cutting member and a drive shaft. The elongated guide tube defines a lumen. The lumen extends through the elongated guide tube from a proximal opening at a proximal end of the elongated guide tube to a distal opening at a distal end of the elongated guide tube. The lumen may also extend linearly over a linear section extending between the bend and the distal opening of the elongated guide tube. The bend can direct the lumen and the distal opening laterally from the longitudinal axis of the elongated guide tube. The inner guide tube is slidably received within the lumen of the elongated guide tube. The cutting head cutting head secured to a distal end of the inner guide tube. The cutting head extends from the distal opening at the distal end of the elongated guide tube. The cutting head defines an anterior cavity at a distal end of the cutting head. The cutting head further includes at least one tissue receiving opening on its distal end. The tissue receiving opening extending from an outer surface of the cutting head to the anterior cavity. The tissue receiving opening receives materials of an intervertebral disc as the cutting head is advanced through the intervertebral disc. The tissue receiving opening may be in the form of one or more slots. The rotary cutting member is positioned within the anterior cavity of the cutting head. The rotary cutting member is configured to cut and/or abrade material received through the tissue receiving opening. The drive shaft extends through an inner guide tube lumen defined by the inner guide tube and is secured to the rotary cutting member to confer rotational movement to the rotary cutting member while positioned within the anterior chamber of the cutting head.

In yet another exemplary embodiment, the present invention may provide an apparatus for removing tissue from an intervertebral disc including an elongated guide tube, a rotary cutting member and a drive shaft. The elongated guide tube defines a lumen. The lumen extends through the elongated guide tube from a proximal opening at a proximal end of the elongated guide tube to a distal opening at a distal end of the elongated guide tube. The lumen includes a bend at the distal end of the elongated guide tube. The bend directs the lumen and the distal opening laterally from the longitudinal axis of the elongated guide tube. The lumen extends linearly over a linear section extending between the bend and the distal opening of the elongated guide tube. The drive shaft is slidably received within the lumen of the elongated guide tube. The rotary cutting member is secured to a distal end of the drive shaft. The rotary cutting member is generally configured to be advanced through a nucleus pulposus of an intervertebral disc to at least one of cut and abrade the nucleus pulposus and to atraumatically contact an annulus fibrosus of the intervertebral disc. The rotary cutting member may be composed of a plurality of filaments configured to cut or abrade tissue. The filaments may have a cross-sectional shape that is round, square, rectangular, parallelogram or other shape as will be recognized by those skilled in the art. The filaments have first ends and second ends. On their second ends, the filaments may include a filament cap. The rotary cutting member may also or alternatively include a plurality of blades to cut or abrade tissue.

In still another exemplary embodiment, the present invention may provide an apparatus for removing tissue from an intervertebral disc including an elongated guide tube, a rotary cutting member and a drive shaft. The elongated guide tube may define a lumen extending from a proximal opening at a proximal end of the elongated guide tube to a distal opening at a distal end of the elongated guide tube. The lumen may include a bend at the distal end of the elongated guide tube. The bend may direct the lumen and the distal opening laterally from the longitudinal axis of the elongated guide tube. The lumen can extend linearly over a linear section extending between the bend and the distal opening. The drive shaft defines a driveshaft lumen and is rotatably received within the lumen of the elongated guide tube. The rotary cutting member defines a proximal recess. The proximal recess extends peripherally around the proximal end of the rotary cutting member. The proximal recess may be received within the driveshaft lumen to secure the rotary cutting member secured to a distal end of the drive shaft. The outer diameter of the rotary cutting member and an outer diameter of the drive shaft may be substantially the same to provide a uniform diameter and profile at the transition between the drive shaft and the rotary cutting member.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

### BRIEF DESCRIPTION OF THE DRAWINGS

FIG. 1 illustrates a perspective view of an embodiment of an apparatus in accordance with the present invention;

FIG. 2 illustrates a cross-section of an embodiment of the distal portion of an apparatus in accordance with the present invention;

FIG. 3 illustrates a side view of an embodiment of a cutting head in accordance with the present invention;

FIG. 4A illustrates an end view of an embodiment of a cutting head in accordance with the present invention;

FIG. 4B illustrates an end view of another embodiment of a cutting head in accordance with the present invention;

FIG. 4C illustrates an end view of yet another embodiment of a cutting head in accordance with the present invention;

FIG. 5A illustrates a side view of cross-section of an embodiment of a cutting head in accordance with the present invention receiving a blade in a collapsed position;

FIG. 5B illustrates a side view of cross-section of an embodiment of FIG. 5A having received the blade and with the blade in an expanded position;

FIG. 6 illustrates a perspective view of an embodiment of an apparatus in accordance with the present invention;

FIG. 7 illustrates a perspective view of another embodiment of an apparatus in accordance with the present invention;

FIG. 8 illustrates a perspective view of an embodiment of a blade for an apparatus in accordance with the present invention;

FIG. 9A illustrates a profile of a cross-section of an embodiment of a blade similar to the blade of FIG. 8;

FIG. 9B illustrates a profile of a cross-section of another embodiment of a blade similar to the blade of FIG. 8;

FIG. 9C illustrates a profile of a cross-section of yet another embodiment of a blade similar to the blade of FIG. 8;

FIG. 9D illustrates a profile of a cross-section of yet another embodiment of a blade similar to the blade of FIG. 8;

FIG. 9E illustrates a profile of a cross-section of still another embodiment of a blade similar to the blade of FIG. 8;

FIG. 10 illustrates a cross-sectional side view of another embodiment of an apparatus in accordance with the present invention;

FIG. 11 illustrates some details of the cutting head in a cross-sectional side view of an embodiment similar to that of FIG. 10;

FIG. 12 illustrates a cross-sectional side view of yet another embodiment of an apparatus in accordance with the present invention; and

FIGS. 13A, 13B and 13C illustrate a sequential series of top views of an embodiment of an apparatus in accordance with the present invention advancing through the nucleus pulposus of an intervertebral disc.

All Figures are illustrated for ease of explanation of the basic teachings of the present invention only; the extensions of the Figures with respect to number, position, relationship and dimensions of the parts to form the preferred embodiment will be explained or will be within the skill of the art after the following description has been read and understood. Further, the exact dimensions and dimensional proportions to conform to specific force, weight, strength, and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings as it would appear to a person viewing the drawings and utilized only to facilitate describing the illustrated embodiment.

### DETAILED DESCRIPTION OF THE INVENTION

The present invention provides an apparatus 10 and methods for removal of materials from an intervertebral disc positioned between adjacent vertebral bodies within the spine of a patient. The apparatus 10 generally provides a rotary cutting member 14 at the distal tip of an elongated guide tube 12 for accessing and removing tissues from an intervertebral disc. The apparatus may also include a cutting head 50 secured about the rotary cutting member 14. The apparatus 10 is generally configured to access the intervertebral disc in a minimally invasive manner. Generally, the rotary cutting member 14 is configured to extend from and retract into the elongated guide tube 12 while rotating to remove or facilitate the removal of tissue from the intervertebral disc. In many procedures, the material removed is tissue from the nucleus pulposus of the intervertebral disc. The apparatus 10 is typically generally configured to permit posterior access to the intervertebral disc wherein elongated guide tube 12 may additionally possess sufficient flexibility to permit the bending of the elongated guide tube 12 around the anatomical structures of the spine.

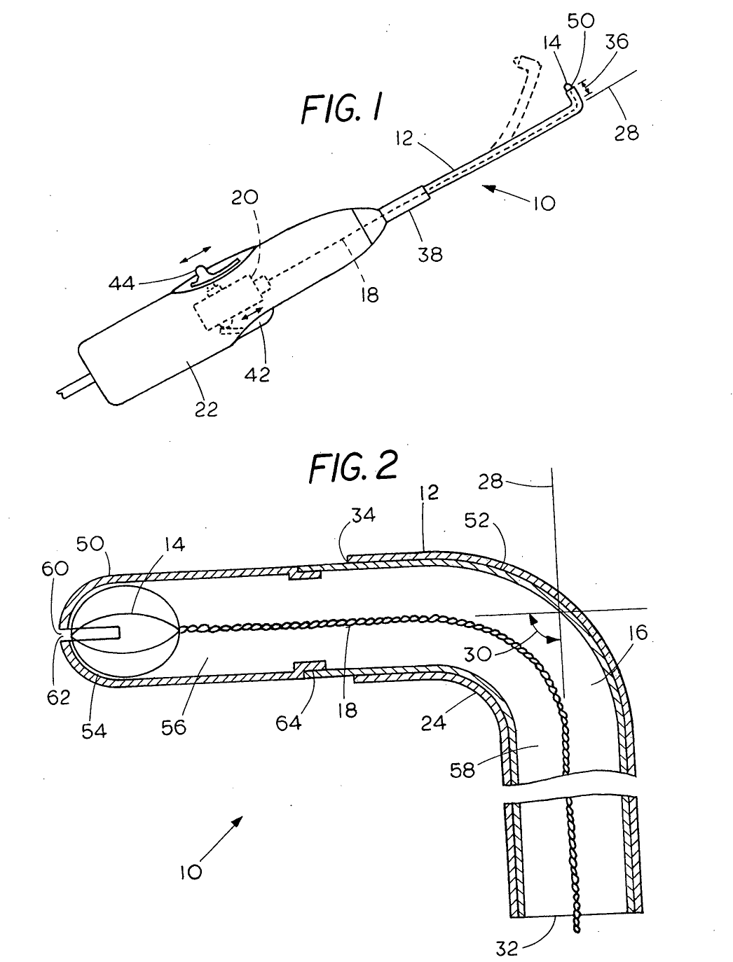

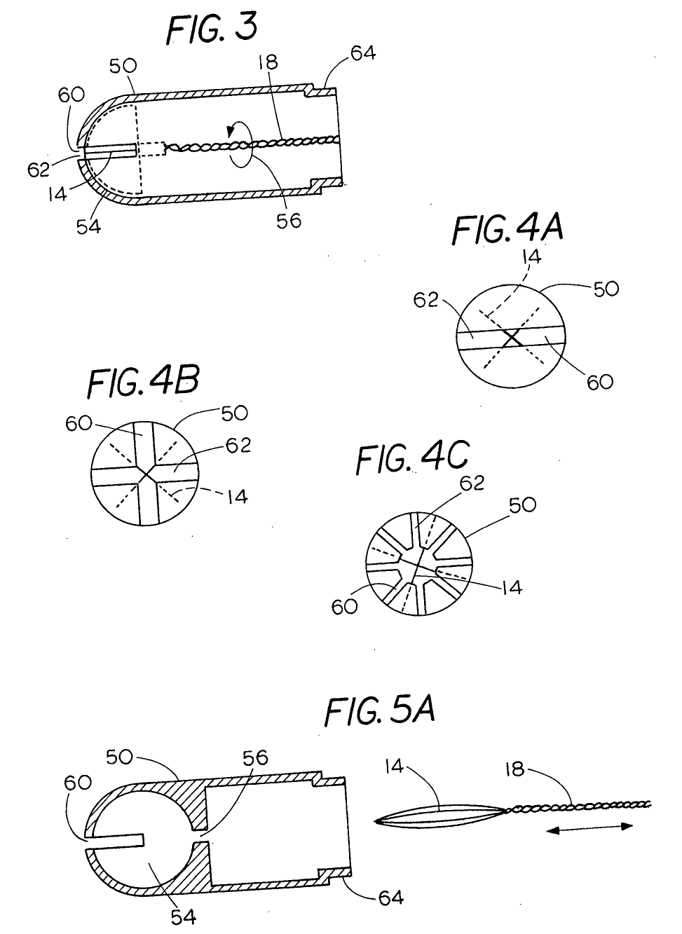

Apparatus 10 in accordance with the present invention generally includes an elongated guide tube 12 having a rotary cutting member 14 as illustrated generally throughout the Figures for exemplary purposes. As illustrated in FIGS. 1 to 5B and 10 to 12, the rotary cutting member 14 may be positioned within an anterior chamber 56 of a cutting head 50. The cutting member 14 and, when present, the cutting head 50 may be extended from or retracted into a lumen 16 defined by the elongated guide tube 12. Typically, the rotary cutting member 14 and the cutting head 50 will be extended and retracted together with the rotary cutting member 14 being retained within an anterior chamber 54 of the cutting head 50 during operation.

A drive shaft 18 is also provided within the lumen 16 of elongated guide tube 12. A distal end of the drive shaft 18 is operably connected to the rotary cutting member 14 to confer a rotatational force upon the rotary cutting member 14. When a cutting head 50 is included, the drive shaft 18 may extend through a posterior passage 56 of the cutting head 50 to connect to the rotary cutting member 14 contained within the anterior chamber 54 of cutting head 50. In one aspect, the drive shaft 18 may rotate the rotary cutting member 14 relative to the anterior chamber 54. The drive shaft 18 is typically operably connected to a motor 20 at a proximal end of the drive shaft 18. However, the drive shaft 18 may be otherwise operably connected to the motor 20 to confer a rotational motion upon the drive shaft as will be recognized by those skilled in the art upon review of the present disclosure. Motor 20 may be an electrical motor, a pneumatic drive system, a hydraulic drive system, or other system or motor as will be recognized by those skilled in the art upon review of the present disclosure. To facilitate the extending and retracting of the rotary cutting member 14, the motor 20 may be movable relative to the elongated guide tube 12. In one aspect, the motor 20 may be slidably mounted in a housing or handle 22 to which the proximal end of elongated guide tube 12 is secured as illustrated in FIG. 1 for exemplary purposes. Housing 22 may be configured to permit a surgeon to grip the housing 22 as a handle to manipulate the distal end of elongated guide tube 12 and/or cutting member 14 to and within an intervertebral disc of a patient. The cutting member 14 may also be movably secured to the distal end of the drive shaft 18 to permit extending and retracting of the cutting member 14, the motor 20 may be movably connected to the proximal end of the drive shaft 18 to permit extending and retracting of the cutting member 14, or the cutting member 14, drive shaft 18 and motor 20 may be otherwise configured to permit extending and retracting of the cutting member 14 as will be recognized by those skilled in the art upon review of the present disclosure. In another aspect, the motor 20 may be provided remotely from the apparatus 10 and transfer the rotational motion to drive shaft 18 through, for example, a transmission and/or clutch assembly 26 located within housing 22. Regardless of configuration, a force is conferred upon the cutting member 14 by a drive shaft 18 having sufficient torque to permit cutting member 14 to cut through the material of the intervertebral disc at a rate sufficient to remove tissue within the time constraints for a particular procedure or a rate preferred by an operating physician.

Elongated guide tube 12 may be configured from a material which permits a surgeon to properly position the distal portion of the elongated guide tube 12 within an intervertebral disc to remove the desired portions of the intervertebral disc. In one aspect, applications may required that the elongated guide catheter 12 have sufficient flexibility to bend and otherwise flex as the distal end of the elongated guide tube 12 is inserted through a patient into the intervertebral disc. In other aspects, applications may require that the elongated guide tube 12 have sufficient stiffness to permit a surgeon to advance the distal end into the intervertebral disc and to precisely maneuver the distal portion of the elongated guide tube 12 within the intervertebral disc. In still other aspects, applications may require that the elongated guide tube 12 have a variable stiffness along its length. Typically, the material used is polymeric such as a high density polyethylene, PTFE, PEBAX, PEEK or other flexible polymeric material which will be recognized by those skilled in the art. However, the material may be a metal, composite materials or other material selected and configured for access to the intervertebral disc. Alternatively, the elongated guide tube 12 may be configured from a stiff material such as a metal to allow precise positioning and movement of the cutting member 14. The elongated guide tube 12 defines a central lumen 16 that extends along the longitudinal axis 28 of the elongated guide tube 12. In one aspect, the central lumen 16 may include a lubricious coating 40 to reduce friction between the walls of lumen 16 and the drive shaft 18. A proximal end of elongated guide tube 12 defines a proximal opening 32 of the lumen 16. The proximal end may be adapted to engage a handle or housing 22, a motor or other structure associated with an apparatus 10. The distal end of the elongated guide tube 12 includes a bend 24 which directs the elongated guide tube 12 and the associated lumen 16 laterally at a desired angle 30 from the longitudinal axis 28. The angle 30 is typically between about 60 degrees and 120 degrees from the longitudinal axis 28. In one aspect, the angle 30 of the bend 24 may be about 90 degrees from the longitudinal axis 28 as is generally illustrated in the figures for exemplary purposes. In other aspects, elongated guide tube 12 may be steerable. One method of providing a steerable feature is for elongated guide tube 12 to possess a second, smaller lumen within the wall of elongated guide tube 12 positioned along the outer radius of the bend 24. A stiff rod or wire can be slidably moved within the smaller lumen, with the effect of straightening the bend 24, at least partially, when the stiff rod or wire is fully inserted along the length of elongated guide tube 12. In this aspect, the degree of bending can be controlled by a user and may be varied during the use of the apparatus 10. The lumen 16 and bend 24 are configured to generally direct the cutting action of rotary cutting member 14 laterally from the longitudinal axis 28. In one aspect, the distal end of elongated guide tube 12 is configured to include linear section 36 of lumen 16 extending laterally from the longitudinal axis 28 between the end of bend 24 and the distal opening 34 to permit the surgeon to orient and linearly advance the rotary cutting member 14 through the material of the intervertebral disc in a desired direction. In applications for extracting materials from an intervertebral disc, the linear section 36 is typically between 0.5 millimeters and 20 millimeters in length.

Rotary cutting member 14 is generally configured to cut, abrade or otherwise disrupt material to permit the concurrent or subsequent removal of tissue. A wide variety of blades, wires, discs and filaments may be used to facilitate the cutting or abrading of material by the rotary cutting member 14 and are illustrated throughout the Figures in various configurations for exemplary purposes. Upon review of the present disclosure, those skilled in the art will recognize additional cutting and abrading configurations for rotary cutting member 14 that may be used in devices in accordance with the present disclosure. The rotary cutting members 14 are typically configured to impart a cutting or abrading action on adjacent tissue when the rotary cutting member 14 is rotated about a central axis. The rotary cutting members 14 in accordance with the present invention are generally configured to be advanced through the tissue of the intervertebral disc from the distal opening 34 of elongated guide tube 12. Typically, the rotary cutting member 14 cuts or abrades tissue as it extends from the distal opening of the guide catheter. Accordingly, the material of the blades is generally selected to withstand the forces conferred by rotational engagement of tissues of the intervertebral disc. Further, the material of the blades may be generally selected to withstand the forces conferred by the surgeon extending and retracting the blade from the lumen of the elongated guide tube 12. In addition, the material for the blades is selected which will not lose its cutting efficiency by, for example, premature dulling in the course of a typical operation. The drive shaft 18 operably couples a motive component conferring rotational movement, such as a motor 20 for example, to the rotary cutting member 14. Drive shafts 18 are frequently in the form of wires, cables, braided wires, coils, and tubes. In one aspect, the drive shaft 18 may define a driveshaft lumen 48 such as may be the case when, for example, a coil is used as a drive shaft 18. A distal end of the drive shaft 18 typically engages the rotary cutting member 14. A drive shaft 18 may, typically at a proximal end, be operably engaged with the motor 20, a transmission and/or clutch assembly 26 connected to a motor 20, or to another rotationally motivating component to confer a rotational force to a rotary cutting member. A drive shaft 18 in accordance with the present invention is typically of a diameter and configuration to be rotatably received within lumen 16 of elongated guide tube 12. Typically, the drive shaft 18 will extend for a length greater than the length of the lumen 16. Such a length can permit the rotary cutting member 14 to be extended beyond the distal opening 34 of lumen 16 to engage a tissue within the intervertebral disc. The drive shafts 18 are typically metals however a range of polymers and other materials may be used as will be recognized by those skilled in the art upon review of the present disclosure.

The cutting head 50 generally contains the rotary cutting member 14. Typically, the rotary cutting member 14 is positioned within an anterior chamber 54 of the cutting head 50. Tissue to be cut and/or abraded by the rotary cutting member is received through a tissue receiving opening 60. Tissue receiving opening 60 is typically positioned at the distal end of the cutting head 50. In one aspect, tissue receiving opening 60 may be configured as a slot 62. The cutting head 50 will typically be constructed from a polymeric material or a metal as will be recognized by those skilled in the art. The cutting head 50 may be sized and shaped to permit the cutting head 50 to be received through lumen 16 of the elongated guide tube 12. The cutting head 50 may further be configured to track in a straight line as the cutting head 50 is extended from the distal opening of elongated guide tube 12. In other applications, the shape of the cutting head 50 may be selected to alter the track of the cutting head 50 as it is advanced through the material of the intervertebral disc. For exemplary purposes, the distal end of cutting head 50 is illustrated with a hemispherical shape. The hemispherical shape of the distal end of cutting head 50 may facilitate a linear tracking as the cutting head 50 is extended from the elongated guide tube 12. In one aspect of the present invention, the cutting head 50 may be generally configured to allow a surgeon to push the cutting head 50 through the tissue of the nucleus pulposus of an intervertebral disc as the rotary cutting member 14 is advanced within the cutting head 50 cutting and/or abrading tissues. Further, the cutting head 50 may be configured to render contact with the more dense and fibrous tissue of the annulus fibrosus atraumatic to that tissue. This may include altering the size and shape of the tissue receiving opening 60 at the distal end of the cutting head 50, may include configuring the shape of the rotary cutting member 14 within the cutting head 50, and/or may include other modifications to the filaments 66 and/or rotary cutting member 14 to render any incidental contact with the annulus fibrosus atraumatic.

FIG. 1 illustrates an exemplary embodiment of an apparatus 10 in accordance with the present invention. As illustrated, apparatus 10 includes a housing 22 in the form of a handle attached to an elongated guide tube 12 including a terminal linear section 36 at a distal end. The housing 22 includes a trigger 42 to actuate the rotation of the motor 20, which for exemplary purposes is slidably secured within the housing 22. An actuator 44 is positioned on a side opposite the trigger 42 on the housing 22. The actuator 44 is operably connected to the motor 20 to slide the motor 20, illustrated in phantom, forward and backwards within the housing 22 as indicated by the arrows. In other embodiments, actuator 44 may be connected to an inner guide tube 52 to motivate the extending and retracting of the rotary cutting member 14. The motor 20 is connected directly to the drive shaft 18 which in turn is engaged with a rotary cutting member 14 within a cutting head 50 positioned adjacent to the distal opening 34 of the lumen 16. In still another embodiment for extending and retracting the rotary cutting member 14 and/or cutting head 50, the elongated guide tube 12 may be slidably received within a sleeve 38 secured to housing 22. Accordingly, the elongated guide tube 12 may be movable relative housing/motor/driveshaft assembly to permit the extending and retracting of the rotary cutting member 14 from distal opening 34 as the elongated guide tube is slid into and out of, respectively, sleeve 38. Elongated guide tube 12 is illustrated in phantom in a flexed position with the flexing initiated at the illustrated point for exemplary purposes.

FIGS. 2 to 5B, 10 and 11 illustrate exemplary embodiments of the distal end of an apparatus 10 in accordance with the present invention. As illustrated, apparatus 10 includes an elongated guide tube 12 and a cutting head 50 containing the rotary cutting member 14. The cutting head 50 is typically secured at a proximal end to a distal end of an inner guide tube 52. The inner guide tube 52 is movably received within lumen 16 of elongated guide tube 12. As illustrated, the inner guide tube 52 may be extended or retracted within the elongated guide tube 12 to extend or retract the cutting head 50 and associated rotary cutting member 14. The guide tube may communicate with the actuator 44 of the housing 22 to allow a user to extend or retract the cutting head 50. Drive shaft 18 extends longitudinally through an inner guide tube lumen 58 defined by the inner guide tube 52 and is secured to the rotary cutting member 14. As illustrated in FIGS. 2 to 5B, the drive shaft 18 further extends through a posterior passage 56 defined by the cutting head 50 to a proximal portion of an anterior chamber 54 defined by the cutting head 50. As illustrated, inner guide tube lumen 58 can be generally coextensive with lumen 16.

As illustrated in FIGS. 2 to 5B, the cutting head 50 defines the anterior chamber 54 which encloses a rotary cutting member 14. The anterior chamber is configured to permit the rotary cutting member 14 to rotate within the anterior chamber 54. Typically, the rotary cutting member 14 will rotate within the cutting head 50 and about a longitudinal axis of the cutting head 50. As illustrated for exemplary purposes in the figures, anterior chamber 54 may be spherical or hemispherical in shape, however other shapes are contemplated in accordance with the present invention.

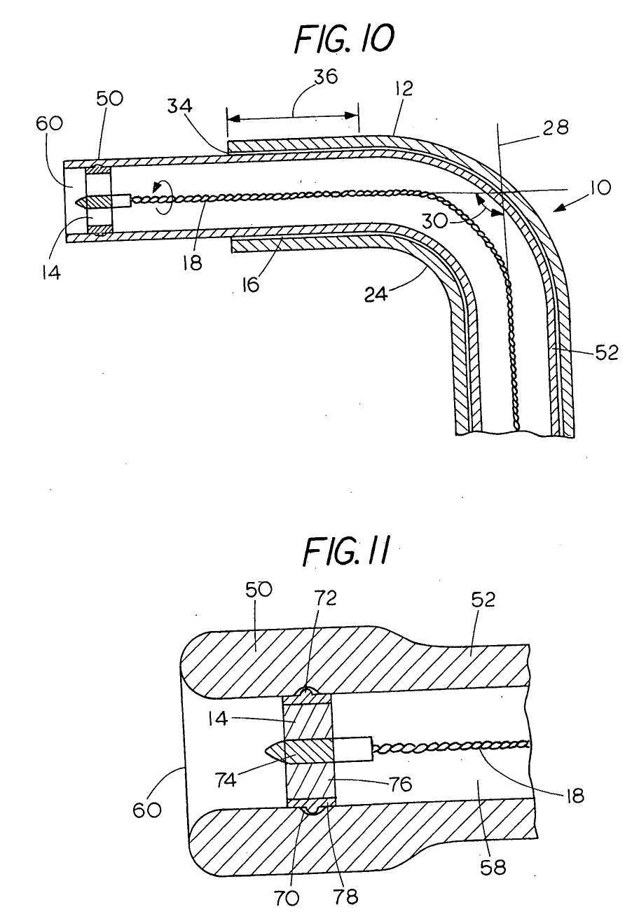

Cutting head 50 includes one or more distal openings 60 extending between an outer surface of the cutting head 50 and the anterior chamber 54. FIGS. 4A, 4B and 4C illustrate some exemplary configurations of openings 60 in the form of slots 62. FIG. 4A illustrates a single slot extending diametrically across the distal portion of cutting head 50. FIG. 4B illustrates two slots extending diametrically across the distal portion of cutting head 50. FIG. 4C illustrates three slots extending diametrically across the distal portion of cutting head 50. The slots 62 are illustrated as extending diametrically across the distal portion of cutting head 50 through the longitudinal axis of cutting head 50 for exemplary purposes. Those skilled in the art will recognize that a range of configurations for slots 62 which will not depart from the scope of the present inventions. For example, a plurality of slots may be positioned in parallel across the distal end of the cutting head 50 or a single slot may be positioned across the distal portion of cutting head 50 without intersecting the longitudinal axis of cutting head 50. FIGS. 10 and 11 illustrated a cutting head 50 defining a substantially circular opening 60 at a distal end of the cutting head 50. Generally, the openings 60 are configured to receive materials of the intervertebral disc as the cutting head is advanced through the intervertebral disc. In one aspect, the configuration of the slots 62 is designed to affect the track of the cutting head 50 as it is advanced through the material of the intervertebral disc. In another aspect, the slot 62 is configured to receive the nucleus pulposus of the intervertebral disc in a manner which permits the adjacent rotary cutting member 14 to cut or ablate the received nucleus pulposus. However, the same configuration of the distal slot 62 may not receive the more dense and fibrous annulus fibrosus about the periphery of the intervertebral disc. Accordingly, this aspect of the present inventions may provide a safety mechanism preventing the blade from extending through the annulus fibrosus of the intervertebral disc in procedures where penetration of the annulus fibrosus is not desired.

As illustrated in FIGS. 10 and 11, the cutting head 50 may rotatably secure the rotary cutting member 14 adjacent to a tissue receiving opening 60 of the cutting head 50. As illustrated, cutting head 50 defines a circumferential groove 70 within a substantially circular opening 60 to slidably receive one or more peripheral protuberances 72 extending from the rotary cutting member 14. The interaction of the circumferential grooves 70 with the peripheral protuberances 72 of the rotary cutting member 14 may function to guide the rotary cutting member 14 in its rotation. Alternatively, the protuberances could extend circumferentially about the opening 60 and the groove could be formed about the periphery of the rotary cutting member 14. Further, grooves could be positioned about both of the opening 60 and the rotary cutting member 14 to receive ball bearings. Upon review of the present disclosure, those skilled in the art will recognize additional configurations for rotatably securing the rotary cutting member 14 adjacent to the opening 60 of the cutting head 50 without departing from the scope of the present invention.

The cutting head 50 may be secured to or integral with the inner guide tube 52 at a distal end of the inner guide tube 52. The inner guide tube 52 generally functions to extend and retract the cutting head to and from the lumen 16 of the elongated guide tube 12. The inner guide tube 52 is illustrated as a wound coil for exemplary purposes other examples may include a tube or a hollow braid among other configurations. The guide tube is typically formed from a metal or polymeric material. The cutting head 50 may be adhesively secured, mechanically secured, welded, compressionally secured, integrally molded or otherwise secured to the guide tube 52. As illustrated in FIGS. 2 to 5B, the cutting head 50 includes a proximal recess 64 about which inner guide tube 52 is secured. The proximal recess 64 may permit the cutting head 50 and inner guide tube 52 to have the same diameter. In one aspect, this may permit the cutting head 50 to be advanced through the tissues of the intervertebral disc without getting caught up or snagged on tissues as the surgeon advances and withdraws the cutting head within an intervertebral disc. The proximal end of the inner guide tube 52 may be connected to actuator 44 or other component to facilitate the movement of the inner guide tube 52 relative to the elongated guide tube 12.

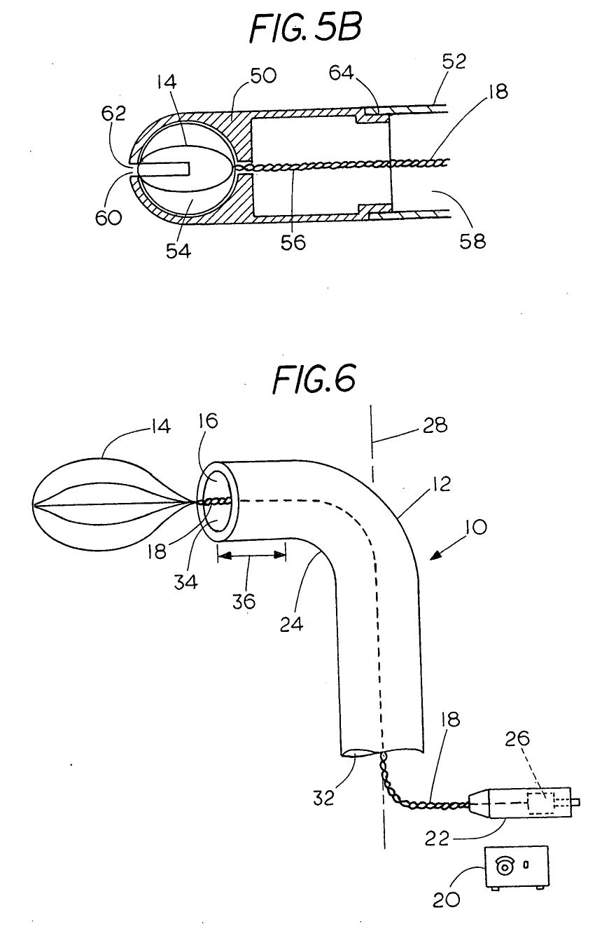

As illustrated in FIGS. 2 to 6 and 9A to 9E, the rotary cutting member 14 may comprise a plurality of filaments 66 secured to the drive shaft 18 at their ends. As illustrated for exemplary purposes in FIGS. 2 to 6 and 9A to 9E, the filaments 66 may be connected at a first end and a second end and assume an ovoid, substantially spherical or substantially hemispherical shape. In other embodiments, the filaments 66 may only be secured to the drive shaft 18 at a first end. When secured at only a first end, the second end may include a filament cap 68 to prevent trauma to the annulus fibrosus or vertebral endplates from cutting or abrasion by the filaments 66. Although typically configured to render contact with the filaments less traumatic or atraumatic, certain configurations of filament caps 68 may enhance cutting and/or abrasion by the filaments. The filaments 66 are typically formed from a metal or a polymeric material. The physical characteristics of the material and the shape and size of the filament 66 as well as the overall configuration of the rotary cutting member 14 will typically dictate the particular cutting or abrading characteristics for a particular rotary cutting member 14. FIGS. 9A, 9B, 9C, 9D and 9E illustrate some exemplary cross-sectional shapes for filaments 66. FIG. 9A illustrates a filament 66 having a circular cross-sectional shape. FIG. 9B illustrates a filament 66 having a square cross-sectional shape. FIG. 9C illustrates a filament 66 having a rectangular cross-sectional shape. FIG. 9D illustrates a filament 66 having a parallelogram cross-sectional shape. FIG. 9E illustrates a filament 66 having a triangular cross-sectional shape. Those skilled in the art will recognize a variety of geometrical and surface configurations that may alter or assist the abrading or cutting action which will not depart from the scope of the present inventions. In one aspect of the present invention, the filaments 66 may be generally configured to allow a surgeon to push the filaments 66 through the tissue of the nucleus pulposus of an intervertebral disc as the rotary cutting member 14 is advanced. Further, the filaments 66 may be configured to render their contact with the more dense and fibrous tissue of the annulus fibrosus would be atraumatic to that tissue. This may include altering the cross-sectional shape of the filaments at the distal end of the rotary cutting member 14, may include configuring the shape of the rotary cutting member 14 at its distal end, and/or may include other modifications to the filaments 66 and/or rotary cutting member 14 to render any incidental contact with the annulus fibrosus atraumatic. In some aspects of the present invention, the rotary cutting member may be collapsible. As illustrated in FIGS. 5A and 5B, this collapsibility may permit the insertion and/or removal of the rotary cutting member 14 to or from the anterior chamber 54 through a posterior passage 56 having a diameter less than the diameter of the rotary cutting member 14.

As illustrated in FIGS. 10 and 11, rotary cutting member 14 may include a plurality of blades 76 radiating out from the axis of rotation of the rotary cutting member 14. As illustrated, the blades 76 may extend from a hub 74 at the axis of rotation to a circumferential ring 78. The blades 76 may be generally configured to receive the tissues of the nucleus pulposus as the blades are advanced through an intervertebral disc.

FIG. 6 illustrates another embodiment of an apparatus 10 in accordance with the present invention. As illustrated, apparatus 10 includes an elongated guide tube 12, a drive shaft 18 and a rotary cutting member 14. The driveshaft 18 is connected at its distal end to a housing 22 including a transmission and/or clutch assembly 26. A remote motor 20 is provided to confer a rotational motion to the driveshaft 18 that is engaged through the transmission and/or clutch assembly 26 using a trigger 42 or button on housing 22. The drive shaft 18 is slidably and rotatably received within lumen 16 of elongated guide tube 12. The drive shaft 18 may be extended or retracted within the elongated guide tube 12 to extend or retract the rotary cutting member 14. As illustrated, the drive shaft 18 may be extended or retracted by moving the housing 22 relative to the elongated guide tube 12. Again, the elongated guide tube 12 is illustrated with a bend 24 at the distal end of the guide catheter. The distal end of elongated guide tube 12 may be further configured to include linear section 36 of lumen 16 extending a sufficient distance between the end of bend 24 and the distal opening 34 to permit the surgeon to orient and linearly advance the rotary cutting member 14 through the material of the intervertebral disc in a desired direction. The rotary cutting member 14 is illustrated as a plurality of filaments 66 secured to the drive shaft 18 at their ends. For exemplary purposes, the filaments 66 are connected at a first end and a second end and assume the substantially ovoid shape shown in FIG. 6. While permitting the cutting and/or abrading of the nucleus pulposus of an intervertebral disc, the filaments 66 may be configured at the distal tip of the rotary cutting member 14 to render the rotational contact of the rotary cutting member with the more dense and fibrous tissue of the annulus fibrosus to be atraumatic. Alternatively, an end cap 86 may be provided at the distal portion of the rotary cutting member 14. The end cap 86 may be generally configured to allow a surgeon to push the end cap through the tissue of the nucleus pulposus of an intervertebral disc as part of the rotary cutting member 14. Further, the end cap 86 may be configured to render the contact by the end cap 86 of the rotary cutting member 14 with the more dense and fibrous tissue of the annulus fibrosus to be atraumatic.

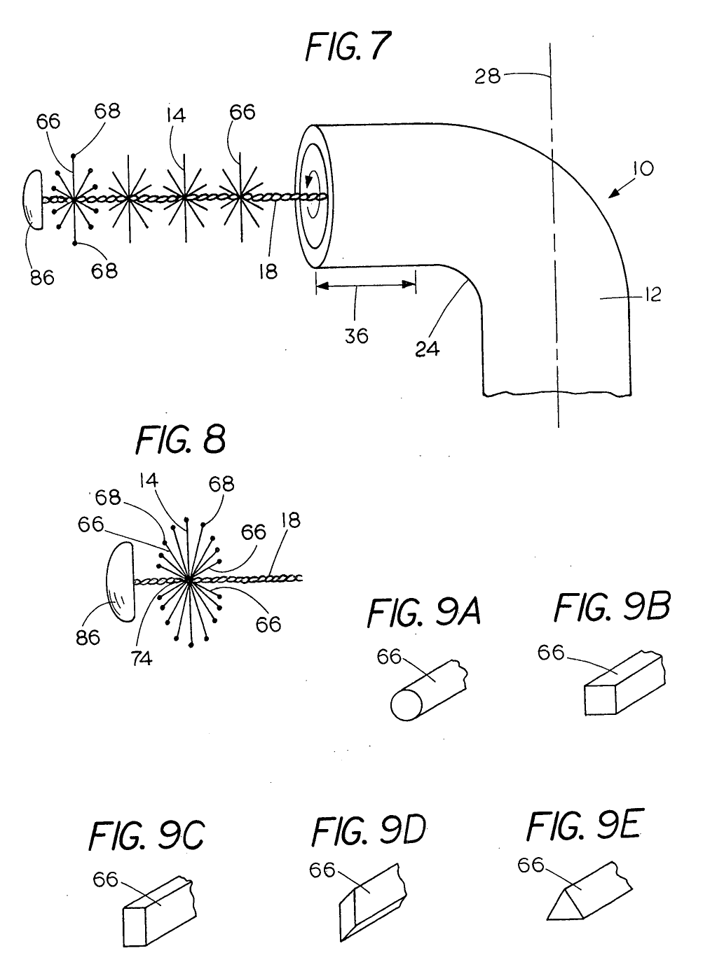

FIGS. 7 and 8 illustrate an embodiment of an apparatus 10 similar to other illustrated embodiments having filaments 66 except that the filaments 66 are secured to the drive shaft 18 only at a first end of the filaments. A hub 74 may be provided to connect the first end of filaments 66 to the drive shaft 18. Again, the filaments 66 are typically formed from a metal or a polymeric material. The physical characteristics of the material and the shape and size of the filament 66 as well as the overall configuration of the rotary cutting member 14 will typically dictate the particular cutting or abrading characteristics for a particular rotary cutting member 14. Again, FIGS. 9A, 9B, 9C, 9D and 9E illustrate some exemplary cross-sectional shapes for filaments 66. FIG. 9A illustrates a filament 66 having a circular cross-sectional shape. FIG. 9B illustrates a filament 66 having a square cross-sectional shape. FIG. 9C illustrates a filament 66 having a rectangular cross-sectional shape. FIG. 9D illustrates a filament 66 having a parallelogram cross-sectional shape. FIG. 9E illustrates a filament 66 having a triangular cross-sectional shape. The filaments 66 of the embodiments of FIGS. 7 and 8 further include caps 86 at their second ends. Caps 86 may be configured to protect the annulus fibrosus and the endplates as the nucleus pulposus is being removed. In addition or alternatively, caps 86 may be provided to enhance the effectiveness of cutting or abrading by filaments 66 including or not including the cutting or abrading of the annulus fibrosus and the endplates. Those skilled in the art will recognize a variety of geometrical and surface configurations for filaments 66 and caps 86 that may alter or assist the abrading or cutting action which will not depart from the scope of the present inventions.

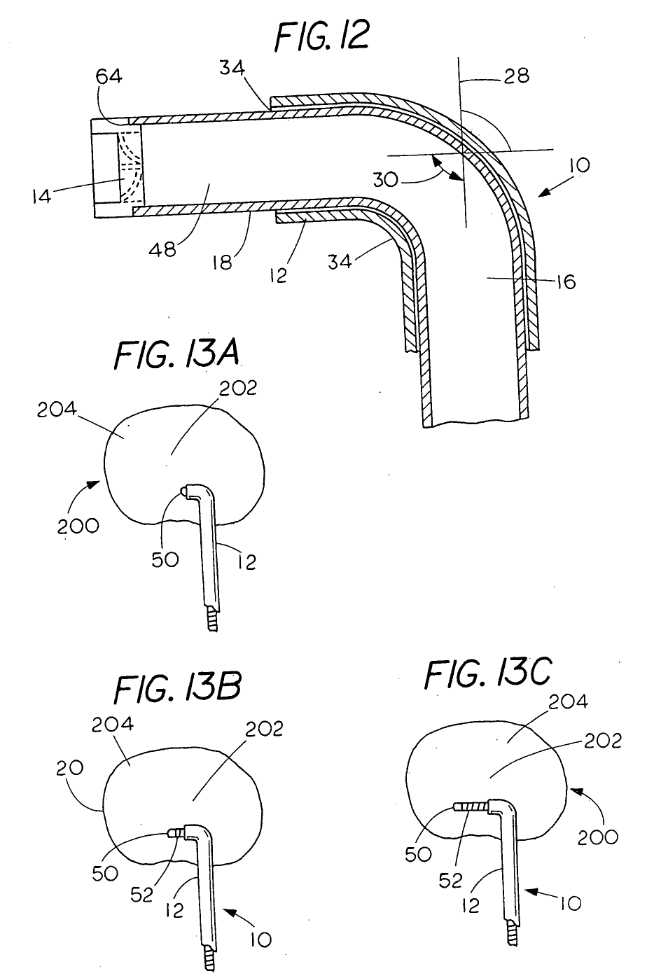

FIG. 12 illustrates an embodiment of an apparatus 10 having a drive shaft 18 in a tubular configuration with an outside diameter approaching the inside diameter of lumen 16. Using such a drive shaft 18, the rotary cutting member may be peripherally secured to the drive shaft 18. When the rotary cutting member includes a proximal recess 64 as illustrated in FIG. 12, the drive shaft 18 and the rotary cutting member 14 may have a uniform diameter. This may better facilitate passing the cutting member 14 and drive shaft 18 through the lumen 16 of the elongated guide tube 12 and may aid in the tracking of the rotary cutting member 14 through the tissues of the intervertebral disc. As illustrated, the drive shaft 18 may provide a drive shaft lumen 48 through which materials may be introduced into or removed from the intervertebral disc.

FIGS. 13A, 13B and 13C illustrate an exemplary sequence for advancing a rotary cutting member 14 enclosed within a cutting head 50 for exemplary purposes through a nucleus pulposus of an intervertebral disc in a de-nucleating procedure. FIG. 1 3A illustrates the elongated guide tube 12 positioned and oriented within the nucleus pulposus of an intervertebral disc with the cutting head 50 retracted within the elongated guide tube 12. FIG. 13B illustrates the cutting head 50 advancing through the nucleus pulposus of the intervertebral disc while receiving material through a slot 62 on the distal end of the cutting head. FIG. 13C illustrates the cutting head having cut a substantially straight track across the nucleus pulposus atraumatically contacting the annulus fibrosus located about the periphery of the intervertebral disc. Once the cutting head 50 has been extended as far as desired, which may be at the periphery of the annulus, the cutting head 50 is retracted into the elongated guide tube 12. More nucleus tissue can be removed by advancing the guide catheter further into the disc cavity and repeating the steps shown in FIGS. 13A-13C. The nucleus material along the opposite side of the elongated guide tube 12 can be removed by rotating the elongated guide tube 12 180 degrees about its long axis and repeating the steps shown in FIGS. 13A-13C while step-wise advancing or withdrawing the elongated guide tube 12 from the disc cavity.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. An apparatus for removing tissue from an intervertebral disc, comprising:
an elongated guide tube defining a lumen extending through the elongated guide tube from a proximal opening at a proximal end of the elongated guide tube to a distal opening at a distal end of the elongated guide tube, the lumen including a bend at the distal end of the elongated guide tube, the bend directing the lumen and the distal opening laterally from the longitudinal axis of the elongated guide tube, the lumen extending linearly over a linear section extending between the bend and the distal opening;
a rotary cutting member slidably received within the distal opening at the distal end of the elongated guide tube; and
a drive shaft rotatably received within the lumen and extending between at least the proximal opening at the proximal end of the elongated guide tube and the distal opening at the distal end of the elongated guide tube, the drive shaft connected to the rotary cutting member to confer a rotational force to the rotary cutting member, and the lumen of the linear section of the elongated guide tube configured to direct the rotary cutting member along an axis defined by the linear section of the elongated guide tube.

2. An apparatus, as in claim 1, wherein the rotary cutting member comprises a plurality of filaments.

3. An apparatus, as in claim 2, further comprising the plurality of filaments having a cross-sectional shape selected from the group of round, square, rectangular, triangular and parallelogram.

4. An apparatus, as in claim 1, wherein the rotary cutting member comprises a plurality of filaments having a first end and a second end and including a filament cap on the second end of at least one of the plurality of filaments.

5. An apparatus, as in claim 1, wherein the rotary cutting member comprises a plurality of blades.

6. An apparatus, as in claim 1, wherein the rotary cutting member comprises an end cap.

7. An apparatus for removing tissue from an intervertebral disc, comprising:
an elongated guide tube defining a lumen extending through the elongated guide tube from a proximal opening at a proximal end of the elongated guide tube to a distal opening at a distal end of the elongated guide tube;
an inner guide tube slidably received within the lumen;
a cutting head secured to a distal end of the inner guide tube and extending from the distal opening at the distal end of the elongated guide tube, the cutting head defining an anterior cavity and having at least one tissue receiving opening on a distal end of the cutting head to receive materials of an intervertebral disc as the cutting head is advanced through the intervertebral disc, the at least one tissue receiving opening extending from an outer surface of the cutting head to the anterior cavity;
a rotary cutting member positioned within the anterior cavity of the cutting head to at least one of cut and abrade material received through the tissue receiving opening; and
a drive shaft extending through an inner guide tube lumen defined by the inner guide tube and secured to the rotary cutting member to confer rotational movement upon the rotary cutting member within the anterior chamber of the cutting head.

8. An apparatus, as in claim 7, further comprising the lumen extending linearly over a linear section extending between a bend and the distal opening of the elongated guide tube, the bend directing the lumen and the distal opening laterally from the longitudinal axis of the elongated guide tube.

9. An apparatus, as in claim 7, further comprising the tissue receiving opening of the cutting head forming a slot.

10. An apparatus, as in claim 7, further comprising the tissue receiving opening of the cutting head forming a plurality of slots.

11. An apparatus for removing tissue from an intervertebral disc, comprising:
an elongated guide tube defining a lumen extending through the elongated guide tube from a proximal opening at a proximal end of the elongated guide tube to a distal opening at a distal end of the elongated guide tube; the lumen including a bend at the distal end of the elongated guide tube, the bend directing the lumen and the distal opening laterally from the longitudinal axis of the elongated guide tube, the lumen extending linearly over a linear section extending between the bend and the distal opening of the elongated guide tube;
a drive shaft slidably received within the lumen of the elongated guide tube; and
a rotary cutting member secured to a distal end of the drive shaft, the rotary cutting member configured to be advanced through a nucleus pulposus of an intervertebral disc to at least one of cut and abrade the nucleus pulposus and to atraumatically contact an annulus fibrosus of the intervertebral disc.

12. An apparatus, as in claim 11, wherein the rotary cutting member comprises a plurality of filaments.

13. An apparatus, as in claim 12, further comprising the plurality of filaments having a cross-sectional shape selected from the group of round, square, rectangular and parallelogram.

14. An apparatus, as in claim 11, wherein the rotary cutting member comprises a plurality of filaments having a first end and a second end and including a filament cap on the second end of at least one of the plurality of filaments.

15. An apparatus, as in claim 11, wherein the rotary cutting member comprises a plurality of blades.

16. An apparatus, as in claim 11, wherein the rotary cutting member comprises an end cap.

17. An apparatus for removing tissue from an intervertebral disc, comprising:
an elongated guide tube defining a lumen extending through the elongated guide tube from a proximal opening at a proximal end of the elongated guide tube to a distal opening at a distal end of the elongated guide tube; the lumen including a bend at the distal end of the elongated guide tube, the bend directing the lumen and the distal opening laterally from the longitudinal axis of the elongated guide tube, the lumen extending linearly over a linear section extending between the bend and the distal opening of the elongated guide tube;
a drive shaft defining a driveshaft lumen rotatably received within the lumen of the elongated guide tube; and
a rotary cutting member defining a proximal recess, the rotary cutting member secured within the driveshaft lumen about the proximal recess to a distal end of the drive shaft.

18. An apparatus, as in claim 17, further comprising an outer diameter of the rotary cutting member and an outer diameter of the drive shaft being substantially the same.
